(19) United States
(12) Patent Application Publication
(10) Pub. No.: US 2026/0137724 A1
(43) Pub. Date: May 21, 2026

(54) COMPOSITION FOR PREVENTING, AMELIORATING OR TREATING LIGAMENT OR TENDON DISEASES

(71) Applicant: Hans Biomed Corp, Seoul (KR)

(72) Inventors: Dae Hyung LEE, Seongnam-si (KR); Mi Ri Kim, Seoul (KR); Ye Ji Choi, Seoul (KR); Da Won Choi, Seoul (KR)

(73) Assignee: Hans Biomed Corp, Seoul (KR)

(21) Appl. No.: 19/392,576

(22) Filed: Nov. 18, 2025

(30) Foreign Application Priority Data
Nov. 18, 2024 (KR) 10-2024-0164296

(51) Int. Cl. A61K 35/32; A61K 9/19; A61K 38/57; A61P 19/04; A61P 29/00

(52) U.S. Cl. CPC A61K 35/32; A61K 9/19; A61K 38/57; A61P 19/04; A61P 29/00

(57) ABSTRACT

Provided is a composition for preventing, ameliorating, or treating ligament or tendon diseases comprising tendon-derived Extracellular Matrix (ECM) powder as an active ingredient, wherein a content of TIMP-1 protein contained in 1 mg of the tendon-derived ECM powder is 500 pg or more.

### TECHNICAL FIEL

The present invention relates to a composition for preventing, ameliorating, or treating ligament or tendon diseases.

### BACKGROUND AR

Tendon and ligament diseases account for 45% of musculoskeletal injuries in modern people and typically refer to diseases such as Achilles tendon disease, patellar tendon disease, rotator cuff tendon disease, tenosynovitis, tendinitis, and tendon rupture.

Specifically, biceps tendinitis and rotator cuff tendinitis most commonly occur in the shoulder area, and appear frequently in workers who work using excessive force or engage in work requiring unnatural working postures such as overhead work, arm-raising work, and bending and lifting arms.

In the case of the elbow region, it is also called lateral epicondylitis or tennis elbow, referring to tendinitis occurring in the elbow due to inflammation of the tendon attached to the muscle located on the outside of the humerus, which can be called the part just above the elbow, and is related to repetitively performing work applying force to the fingers, wrists, and arms.

In addition, examples include flexor tendinitis which is inflammation of the tendon on the palmar surface of the wrist and hand, extensor tendinitis which is inflammation of the tendon on the dorsal surface of the wrist and hand, flexor tenosynovitis which is inflammation of the tendon sheath on the palmar surface of the wrist and hand, extensor tenosynovitis which is inflammation of the tendon sheath on the dorsal surface of the wrist and back of the hand, De Quervain's disease which is inflammation of the tendon sheath at the base of the thumb, and Dupuytren's contracture which is a disease in which fibrosis progresses in the subcutaneous tissue of the palm and mainly causes flexion contracture of the proximal phalanx.

Tendons and ligaments are fibrous soft tissues and have collagen as a main component, and differ only in attachment points to bone-to-bone and bone-to-muscle, respectively, but are similar not only in mechanical properties but also in structural aspects.

Among human tissues, tendons or ligaments have relatively insufficient blood flow supply compared to other tissues of the human body, so once damaged, it takes a considerable amount of time to regenerate, and even if regenerated and treated, it is known that their functions are not completely recovered like normal tendons or ligaments.

It has been reported that even after regeneration, their biomechanical strength is lower than that of normal tendons or ligaments, and such biomechanical strength is influenced by collagen constituting these tissues.

Meanwhile, studies on various growth factors affecting the treatment of tendons and ligaments have also been reported (Molly T. et al. (2003) The roles of growth factors in tendon and ligament healing. Sports Medicine 33, 5: 381-394).

Damage due to trauma to tendons or ligaments commonly occurs in sports, work, or daily life, and inflammation or partial rupture of tendons due to degenerative changes caused by aging can occur even with minor trauma.

However, until now, for tendons or ligaments damaged by inflammation, partial rupture (sprain), or complete rupture of tendons due to such aging (degenerative changes) and trauma, no special treatment method has been developed other than surgical treatment to suture severed tendons or ligaments, steroid injections which only alleviate symptoms, and physical therapy.

Therefore, there is a demand for treatment of tendon and ligament diseases capable of substantially ameliorating or treating tendon or ligament diseases by regenerating and repairing damaged tendons.

Recently, regarding such treatment of tendon and ligament diseases, regenerative treatment using proteins or stem cells is being actively studied.

Patent Document 1 discloses a composition comprising autologous and allogeneic adipose-derived mesenchymal stem cells for healing tendon and ligament damage and a method for manufacturing the same.

The above document reveals that when a composition comprising autologous and allogeneic adipose-derived mesenchymal stem cells is administered, collagen, extracellular matrix (ECM) proteins, and various growth factors are secreted to heal tendon and ligament damage, but there is a technical limitation in that the effect is not continuous when the composition is administered once.

Patent Document 2 describes that tendon and ligament diseases can be treated by administering a composition comprising platelet-derived growth factor (PDGF), but the platelet-derived growth factor has a very low economic feasibility because the process of separation from biological fluids including blood is complicated and obtaining it using recombinant DNA techniques also requires several processes.

In addition, there is a problem that when a substance that is not an artificial substance or an allogeneic derived substance is applied to clinical practice, it cannot be guaranteed that it will be perfectly engrafted in the subject and perform the expected function without side effects.

Although the above prior arts suggest methods for treating tendon and ligament diseases by regulating differentiation of stem cells or administering growth factors to tendon and ligament disease sites, it is difficult to confirm that they can exhibit clear effects without side effects unless they are autologous and allogeneic derived cells or substances, and economic feasibility is low when complicated steps are required.

Therefore, research on new treatment methods capable of effectively regenerating tendon and ligament tissues is required.

### Prior Art Document

### Patent Document

(Patent Document 1) Korean Patent Publication No. 2016-0105363
(Patent Document 2) Korean Patent Publication No. 2013-0000397

### SUMMARY OF THE INVENTION

### Technical Problem

The present invention is intended to solve the problems of the prior art described above, and an aspect of the present invention is to provide a composition for preventing, ameliorating, or treating ligament or tendon diseases having excellent regeneration effects and inflammation and pain reduction effects.

In addition, another aspect of the present invention is to provide a composition for preventing, ameliorating, or treating ligament or tendon diseases with improved usability and biological safety.

### Technical Solution

According to one aspect, there is provided a composition for preventing, ameliorating, or treating ligament or tendon diseases comprising tendon-derived Extracellular Matrix (ECM) powder, wherein a content of TIMP-1 protein contained in 1 mg of the tendon-derived ECM powder is 500 pg or more.

In one embodiment, a crude fat content in the tendon-derived ECM powder may be 2.0% by weight or less based on 100% by weight of the tendon-derived ECM powder.

In one embodiment, an average particle diameter of the tendon-derived ECM powder may be 425 μm or less.

In one embodiment, the tendon-derived ECM powder may be included in an amount of 1 to 99% by weight relative to 100% by weight of the total composition.

In one embodiment, the tendon-derived ECM powder may be manufactured by a method comprising:
(a) collecting tendon tissue;
(b) decellularizing the tendon tissue to obtain tendon-derived ECM;
(c) freeze-drying the tendon-derived ECM;
(d) defatting the freeze-dried tendon-derived ECM; and
(e) degassing the defatted tendon-derived ECM, followed by freeze-grinding to obtain tendon-derived ECM powder.

In one embodiment, the composition for preventing, ameliorating, or treating ligament or tendon diseases may further comprise one or more biocompatible polymers selected from the group consisting of hyaluronic acid, polynucleotide, polydeoxyribonucleotide, carboxymethyl cellulose, collagen, chitosan, alginate, and gelatin.

In one embodiment, the biocompatible polymer may be cross-linked with one or more cross-linking agents selected from the group consisting of 1,4-butanediol diglycidyl ether (BDDE), ethylene glycol diglycidyl ether (EGDGE), 1,6-hexanediol diglycidyl ether, propylene glycol diglycidyl ether, polypropylene glycol diglycidyl ether, polytetramethylene glycol diglycidyl ether, neopentyl glycol diglycidyl ether, and 1,2-(bis(2,3-epoxypropoxy)ethylene).

In one embodiment, the composition for preventing, ameliorating, or treating ligament or tendon diseases may have a pH of 5.0 to 8.0.

### Advantageous Effects

The composition for preventing, ameliorating, or treating ligament or tendon diseases according to the present invention is excellent in regeneration, inflammation, and pain relief effects, and has the advantage of high user satisfaction.

The composition for preventing, ameliorating, or treating ligament or tendon diseases according to the present invention has the advantage that the amelioration effect can be maximized by supplying and supplementing components homogeneous to the original tendon directly to the tendon tissue disease site to create the most suitable environment for amelioration of damaged ligament tendon tissue.

The composition for preventing, ameliorating, or treating ligament or tendon diseases according to the present invention has the advantage that ligament tendon tissue can be safely and effectively ameliorated and regenerated by minimizing residue of immune response inducing factors.

The effects of the present invention are not limited to the above-described effect and should be understood to include all effects that can be inferred from the configuration described in the detailed description or claims of the present invention.

### BRIEF DESCRIPTION OF THE DRAWINGS

FIGS. 1(a), 1(b) and 1(c) are results of analyzing a total of 48 types of growth factors for ECM powders of Preparation Example 2, Comparative Preparation Example 5, and Comparative Preparation Example 6, respectively.

FIG. 2 is a graph showing TIMP-1 content for human tendon-derived ECM powders of Comparative Preparation Examples 1 to 4 and Preparation Examples 1 to 3.

FIG. 3 is a graph showing crude fat residual amount for human tendon-derived ECM powders of Comparative Preparation Examples 1 to 4 and Preparation Examples 1 to 3.

FIG. 4 is a graph showing biological safety test results for compositions comprising human tendon-derived ECM powders of Comparative Preparation Examples 1 to 4 and Preparation Examples 1 to 3.

FIGS. 5(a) and 5(b) are results of analyzing a total of 36 types of inflammation-related factors for human-derived fibroblast culture medium before and after treatment with the composition comprising human tendon-derived ECM powder of Preparation Example 2, respectively.

FIG. 6 is a graph illustrating changes in content of pro-inflammatory factors and anti-inflammatory factors before and after treatment with the composition comprising human tendon-derived ECM powder of Preparation Example 2.

FIG. 7 is a graph showing a degree of inhibition on MMP-1.

FIG. 8 is a graph showing a degree of inhibition on MMP-3.

### DETAILED DESCRIPTION OF THE INVENTION

Hereinafter, the present invention will be described based on specific examples. However, the description of the present invention may be implemented in various different forms, and thus is not limited to the embodiments described herein.

### Definitions

Throughout the specification, when a part “comprises” a certain component, it means that it may further include other components, not excluding other components, unless specifically stated to the contrary.

When a range of numerical values is described throughout the specification, unless the specific range thereof is otherwise stated, the value has the precision of the significant figures provided according to the standard rules in chemistry for significant figures.

For example, 10 includes the range of 5.0 to 14.9, and the number 10.0 includes the range of 9.50 to 10.49.

Throughout the specification, the term “in vivo” refers to events occurring in the body of a subject. Throughout the specification, the term “ex vivo” refers to events occurring outside the body of a subject.

Ex vivo analysis includes cell-based analysis in which living or dead cells are used, and may include cell-free analysis in which intact cells are not used.

Throughout the specification, the terms “prevention”, “amelioration”, and “treatment” are used interchangeably and include effects such as preventing, inhibiting, delaying, improving, resolving, alleviating, relieving, ameliorating, and treating ligament or tendon diseases, and are not necessarily limited to complete inhibition of the occurrence of ligament or tendon diseases.

Throughout the specification, when referred to as “comprising as an active ingredient”, it means that the corresponding ingredient is included in an amount necessary or sufficient to realize a desired biological effect.

In actual application, the determination of the amount included as an active ingredient is an amount for treating a target disease, and may be determined in consideration of matters that do not cause other toxicities, and may vary depending on various factors such as, for example, the disease or condition to be treated, the form of the composition to be administered, the size of the subject, or the severity of the disease or condition.

A person skilled in the art to which the present invention pertains can empirically determine the effective amount of individual compositions without accompanying excessive experimentation.

Throughout the specification, the term “pharmaceutically acceptable carrier” may be one or more selected from the group consisting of saline, sterile water, Ringer's solution, buffered saline, dextrose solution, maltodextrin solution, glycerol, and ethanol, and may further comprise other conventional additives such as antioxidants, buffers, and bacteriostatic agents as necessary.

In addition, diluents, dispersants, surfactants, binders, and lubricants may be additionally added to formulate into injectable formulations such as aqueous solutions, suspensions, emulsions, etc., pills, capsules, granules, or tablets.

Throughout the specification, “tendon tissue” refers to allogeneic or xenogeneic derived tendon tissue, where “allogeneic” refers to human, and “xenogeneic” refers to mammals other than human, such as pigs, horses, cows, sheep, dogs, and rodents.

However, since xenogeneic derived tendon tissue has a concern of animal-derived viruses and zoonotic infection, allogeneic derived tendon tissue may be more preferable for application to the human body.

Throughout the specification, the term “decellularization” refers to general (80% or more), nearly complete (95% or more), or essentially complete (99% or more) removal of cellular components of tendon tissue.

Throughout the specification, the term “subject” may be a mammal such as a rat, livestock, mouse, human, etc., and preferably may be a human.

### Composition for Preventing, Ameliorating, or Treating Ligament or Tendon Diseases

The composition for preventing, ameliorating, or treating ligament or tendon diseases, which is one aspect of the present invention, comprises tendon-derived ECM powder as an active ingredient.

Extracellular matrix (ECM) is mainly responsible for structural support of animals, etc., and 90% or more of the components are collagen which is a structural protein, and the remaining 10% is composed of fibronectin which is a glycoprotein, laminin, glycosaminoglycan (GAG), etc.

In addition, it is known that growth factors and some cytokines exist to promote cell growth and tissue-specific differentiation of undifferentiated cells.

Since such growth factors and cytokines also contain anti-inflammatory factors and pain-controlling factors, they can play an important role in pain relief and treatment. Specifically, they include TIMP-1 (tissue inhibitors of metalloproteinases-1), TIMP-4, etc., which are representative cytokines secreted in relation to pain and inflammation relief, and these cytokines are known to play a role in inducing suppression of inflammatory responses and inducing suppression of inflammatory cytokines.

As a conventional therapeutic agent using the advantages of ECM, there is acellular dermal matrix, which is a human-derived material obtained by subjecting human skin tissue donated after death only to a decellularization process, and ECM is the main component.

Acellular dermal matrix was first developed by being applied to burn patients during the period when emergency medicine was developing in the 1970s, and is currently used for various tissue regeneration and reconstruction purposes such as trauma, ulcers, abdominal wall reconstruction, breast reconstruction, vocal cord paralysis plastic surgery, interdental papilla transplantation, etc. in addition to burns, and in particular, is widely used for wound dressings and artificial skin.

Acellular dermal matrix products are used in sheet type and liquid type of particle or particulate form.

Meanwhile, it is known that ECM extracted from different tissues has tissue specificity having characteristics of each tissue, so that cytokines or growth factors suitable for the tissue are attached to create the most suitable environment for cells to live and constitute each tissue.

For this reason, faster tissue regeneration can be expected if the original components are supplemented to the damaged tissue.

Accordingly, the present inventors have conducted intensive studies to manufacture a powder by freeze-drying and finely grinding the ECM obtained after the decellularization process from tendon tissue and provide the powder as a composition of a paste formulation with high usability. As a result, they found that when TIMP-1 protein present in the tendon-derived ECM is well preserved in the process of powdering the tendon-derived ECM, excellent regeneration effects and inflammation and pain reduction effects are obtained, and thus completed the present invention.

That is, the composition for preventing, ameliorating, or treating ligament or tendon diseases, which is one aspect of the present invention, comprises tendon-derived ECM powder, characterized in that the content of TIMP-1 protein contained in 1 mg of the tendon-derived ECM powder is 500 pg or more.

For example, it may be 500 pg, 600 pg, 700 pg, 800 pg, 900 pg, 1,000 pg, 1,500 pg, 2,000 pg, 2,500 pg, 3,000 pg, 3,500 pg, 4,000 pg, 4,500 pg, 5,000 pg, 5,500 pg, 6,000 pg, 6,500 pg, 7,000 pg, 7,500 pg, 8,000 pg, 8,500 pg, 9,000 pg, 9,500 pg, 10,000 pg, 11,000 pg, 12,000 pg, 13,000 pg, 14,000 pg, 15,000 pg, 16,000 pg, 17,000 pg, 18,000 pg, 19,000 pg, 20,000 pg, 21,000 pg, 22,000 pg, 23,000 pg, 24,000 pg, 25,000 pg, 26,000 pg, 27,000 pg, 28,000 pg, 29,000 pg, 30,000 pg, 31,000 pg, 32,000 pg, 33,000 pg, 34,000 pg, 35,000 pg, 36,000 pg, 37,000 pg, 38,000 pg, 39,000 pg, 40,000 pg, 41,000 pg, 42,000 pg, 43,000 pg, 44,000 pg, 45,000 pg, 46,000 pg, 47,000 pg, 48,000 pg, 49,000 pg, 50,000 pg, or a value between these two values, but is not limited thereto.

According to one example, the crude fat content in the tendon-derived ECM powder may be 2.0% by weight or less, preferably 1.8% by weight or less, more preferably 1.5% by weight or less, and most preferably 1.0% by weight or less, based on 100% by weight of the tendon-derived ECM powder.

When the crude fat content in the tendon-derived ECM powder is controlled within the above range, residue of immune response inducing factors is minimized, so that biological safety can be maximized.

According to one example, the average particle diameter of the tendon-derived ECM powder may be 425 μm or less, preferably 300 μm or less, more preferably 150 μm or less, and most preferably 100 μm or less.

When the average particle diameter of the tendon-derived ECM powder is controlled within the above range, needle discharge is possible, so that convenience of use can be increased.

Meanwhile, since the smaller the average particle diameter of the tendon-derived ECM powder, the more advantageous in terms of convenience of use, the lower limit thereof is not particularly limited in the present invention.

According to one example, the tendon-derived ECM powder may be included in an amount of 0.1 to 90% by weight, preferably 0.5 to 70% by weight, more preferably 0.8 to 50% by weight, even more preferably 1 to 30% by weight, and most preferably 3 to 15% by weight, relative to 100% by weight of the total composition.

When the content of the tendon-derived ECM powder is controlled within the above range, inflammation and pain relief efficacy can be maximized.

The tendon-derived ECM powder containing a high content of TIMP-1 protein as described above can be manufactured by various methods, and the manufacturing method is not particularly limited.

However, as a preferred example, the tendon-derived ECM powder may be manufactured by a method comprising: (a) collecting tendon tissue; (b) decellularizing the tendon tissue; (c) freeze-drying the tendon-derived ECM; (d) defatting the freeze-dried tendon-derived ECM; and (e) degassing the defatted tendon-derived ECM, followed by freeze-grinding to obtain tendon-derived ECM powder.

Since detailed reaction conditions in each step are known in the art, they will not be described separately herein, and the detailed manufacturing method may refer to Preparation Examples to be described later.

However, the present invention is different from the prior arts in that the freeze-dried tissue from which moisture has been removed is defatted and degassed to be powdered.

In this case, there is an advantage that not only pain relief-related proteins and various growth factors supported in the ECM powder remain more effectively, but also biological safety is increased, making it more effective for preventing, ameliorating, or treating ligament or tendon diseases.

The composition for preventing, ameliorating, or treating ligament or tendon diseases of the present invention may further comprise a biocompatible polymer, and the biocompatible polymer may be one or more selected from the group consisting of hyaluronic acid, polynucleotide, polydeoxyribonucleotide, and carboxymethyl cellulose, but is not limited thereto.

Hyaluronic acid (Sodium hyaluronate) is a viscous polymer substance used for protection of cells, maintenance of tissue gaps, and tissue lubrication, and refers to a composition that is a long-chain glycosaminoglycan composed of N-acetylglucosamine and glucuronic acid, has a hydrophilic group of a very large molecular weight, and exhibits high viscosity.

Hyaluronic acid has excellent biocompatibility, does not have interspecies, inter-tissue, and organ specificity, and acts like a lubricant to facilitate the movement of collagen between cells at the damaged site in case of tissue damage.

Many studies are being conducted to form hydrogels through cross-linking using chemical cross-linking agents in order to maintain and supplement the viscosity and strength of hyaluronic acid.

As a general method for obtaining hyaluronic acid, a method of extracting from umbilical cord and tissue, etc., a method of extracting and purifying from cultured bacteria, or commercially available ones for medical use may be purchased and used.

Polynucleotide (PN) forms a chain-shaped polynucleotide by covalently linking the 3rd carbon of pentose constituting a nucleotide and a phosphate group of another nucleotide.

It refers to one or more types or a DNA or RNA strand of a certain length or more.

This does not depend on effective effects according to sequence, and includes random polynucleotide mixtures regardless of sequence.

Polynucleotide promotes the generation of ECM, which is an intercellular constituent material, and plays a role in activating skin healing ability in the human body, thereby functioning to restore the skin's own aging and atrophied regeneration ability.

Polynucleotide is also used as a filler for supplementation and regeneration of skin tissue in conventional aesthetic medicine, and has also been found to be effective in alleviating pain in knee osteoarthritis.

A polynucleotide cross-linked product in which polynucleotides are physically or chemically bonded to each other via a linker, and a polynucleotide cross-linked product gel composition comprising a bioactive substance dispersed in the cross-linked product may be provided.

The polynucleotide may be characterized by being derived from one or more selected from the group consisting of animals, plants, and microorganisms.

For example, it may be separated and/or extracted from semen and/or testis of salmon, which is a fish, and commercially available ones for medical use may be purchased and used.

Polydeoxyribonucleotide (PDRN) is a nucleic acid fragment having one or more types of length, that is, a DNA fragment mixture.

This does not depend on effective effects according to sequence, and includes random polydeoxyribonucleotide mixtures regardless of sequence.

Polydeoxyribonucleotide acts as a signaling substance indicating that there is surrounding cell damage, and is known to play a role in helping recovery and regeneration of damaged sites by activating regeneration mechanisms in the body.

It promotes the generation of ECM, which is an intercellular constituent material, and plays a role in activating skin healing ability in the human body, thereby functioning to restore the skin's own aging and atrophied regeneration ability.

In addition, it exhibits regeneration effects through cell proliferation along with anti-inflammatory effects. For this reason, polydeoxyribonucleotide is utilized for treatment of tissue regeneration after tissue transplantation, arthritis, etc., in the prior art, and is known as a medicine without special side effects because the nucleic acid fragment itself is a biological constituent material.

The polydeoxyribonucleotide may be characterized by being derived from one or more selected from the group consisting of animals, plants, and microorganisms.

For example, it may be separated and/or extracted from semen and/or testis of salmon, which is a fish, and commercially available ones for medical use may be purchased and used.

Carboxymethyl cellulose (CMC) refers to one in which the hydroxyl group of glucose constituting it is substituted with a carboxymethyl group.

Such carboxymethyl cellulose is used in various fields such as glue, food, cosmetics, pharmaceutical additives, etc., and is widely used as a medical material due to its excellent biocompatibility.

Carboxymethyl cellulose functions as a high-viscosity carrier, and has the property of gelling upon contact with body fluids, so it maintains its shape without decomposing for a certain period of time, buffering and supplementing the space between tissues, and can supply effective factors effective for wound regeneration.

Such carboxymethyl cellulose may be separated and/or extracted, and commercially available ones for medical use may be purchased and used.

In addition, the composition for preventing, ameliorating, or treating ligament or tendon diseases of the present invention may further comprise one or more hydrophilic natural polymers selected from the group consisting of collagen, chitosan, alginate, and gelatin as a biocompatible polymer, but is not limited thereto.

According to one example, the biocompatible polymer may be cross-linked with one or more cross-linking agents selected from the group consisting of 1,4-butanediol diglycidyl ether (BDDE), ethylene glycol diglycidyl ether (EGDGE), 1,6-hexanediol diglycidyl ether, propylene glycol diglycidyl ether, polypropylene glycol diglycidyl ether, polytetramethylene glycol diglycidyl ether, neopentyl glycol diglycidyl ether, and 1,2-(bis(2,3-epoxypropoxy)ethylene).

When the composition for preventing, ameliorating, or treating ligament or tendon diseases of the present invention further comprises a biocompatible polymer, the content thereof may be 1 to 99% by weight, preferably 70 to 99% by weight, and more preferably 85 to 97% by weight, relative to 100% by weight of the total composition.

The pH of the composition for preventing, ameliorating, or treating ligament or tendon diseases of the present invention may be 5.0 to 8.0.

The pH of the composition may be adjusted using a separate pH adjusting agent such as citric acid, sodium citrate, malic acid, sodium malate, fumaric acid, sodium fumarate, succinic acid, sodium succinate, sodium hydroxide, sodium hydrogen phosphate, or may be adjusted through weight ratio control of each component.

The composition for preventing, ameliorating, or treating ligament or tendon diseases of the present invention may further comprise pharmaceutically acceptable additives conventionally used in the manufacture of medical compositions.

Pharmaceutically acceptable additives refer to carriers or diluents that do not significantly irritate the organism and do not inhibit the biological activity and properties of the administered compound.

In addition, the additives may improve the manufacture, compressibility, appearance, and taste of the formulation, and for example, stabilizers, surfactants, lubricants, solubilizers, buffers, sweeteners, bases, adsorbents, flavor correctives, binders, suspending agents, hardeners, antioxidants, polishing agents, fragrances, flavoring agents, pigments, coating agents, wetting agents, humidity regulators, fillers, defoaming agents, refreshing agents, masticatories, antistatic agents, coloring agents, sugar coating agents, isotonic agents, softeners, emulsifiers, adhesives, thickeners, foaming agents, pH adjusters, excipients, dispersants, disintegrants, waterproofing agents, antiseptics, preservatives, dissolution aids, solvents, glidants, etc. may be added as necessary.

The composition for preventing, ameliorating, or treating ligament or tendon diseases of the present invention may be formulated and used in various forms according to conventional methods, and for example, may be formulated and used in the form of aqueous solutions, suspensions, emulsions, pastes, gels, powders, and injections.

The composition for preventing, ameliorating, or treating ligament or tendon diseases of the present invention may be formulated in various forms for administration to a subject, and a representative formulation for parenteral administration is an injection formulation, and an isotonic aqueous solution or suspension is preferable.

Injection formulations may be prepared according to techniques known in the art using suitable dispersants or wetting agents and suspending agents.

For example, each component may be dissolved in saline or buffer to be formulated for injection.

Hereinafter, Examples of the present invention will be described in more detail. However, the following experimental results describe only representative experimental results among the above Examples, and the scope and content of the present invention cannot be interpreted as being reduced or limited by the Examples, etc.

Each effect of the various embodiments of the present invention not explicitly presented below will be specifically described in the corresponding section.

### Comparative Preparation Example 1

Soft tissue of human tendon was removed, washed with purified water, and then pretreated by a freeze-drying process.

The freeze-dried tendon tissue was freeze-ground and sieved through a sieve having an opening size of 75 to 425 μm.

The grinding and sieving processes were repeated several times to obtain finely ground human tendon-derived ECM powder.

### Comparative Preparation Example 2

Finely ground human tendon-derived ECM powder was obtained in the same manner as in Comparative Preparation Example 1, except that human tendon from which soft tissue was removed was washed with purified water, then subjected to a defatting process once before freeze-drying, and a degassing process was performed after freeze-drying and before freeze-grinding.

### Comparative Preparation Example 3

Finely ground human tendon-derived ECM powder was obtained in the same manner as in Comparative Preparation Example 1, except that human tendon from which soft tissue was removed was washed with purified water, then subjected to a defatting process twice before freeze-drying, and a degassing process was performed after freeze-drying and before freeze-grinding.

### Comparative Preparation Example 4

Finely ground human tendon-derived ECM powder was obtained in the same manner as in Comparative Preparation Example 1, except that human tendon from which soft tissue was removed was washed with purified water, then subjected to a defatting process three times before freeze-drying, and a degassing process was performed after freeze-drying and before freeze-grinding.

### Preparation Example 1

Finely ground human tendon-derived ECM powder was obtained in the same manner as in Comparative Preparation Example 1, except that a defatting process was performed once and a degassing process was performed before freeze-grinding the freeze-dried tendon tissue.

### Preparation Example 2

Finely ground human tendon-derived ECM powder was obtained in the same manner as in Comparative Preparation Example 1, except that a defatting process was performed twice and a degassing process was performed before freeze-grinding the freeze-dried tendon tissue.

### Preparation Example 3

Finely ground human tendon-derived ECM powder was obtained in the same manner as in Comparative Preparation Example 1, except that a defatting process was performed three times and a degassing process was performed before freeze-grinding the freeze-dried tendon tissue.

### Comparative Preparation Example 5

The epidermis of human skin was removed and washed with a washing solution, followed by a decellularization process. The decellularized skin tissue was pretreated by a freeze-drying process.

The freeze-dried skin tissue was freeze-ground and sieved through a sieve having an opening size of 75 to 425 μm.

The grinding and sieving processes were repeated several times to obtain finely ground human skin-derived ECM powder.

### Comparative Preparation Example 6

Soft tissue of human costal cartilage was removed and washed with a washing solution, and then pretreated by a freeze-drying process.

The freeze-dried costal cartilage tissue was freeze-ground and sieved through a sieve having an opening size of 75 to 425 μm.

The grinding and sieving processes were repeated several times to obtain finely ground human costal cartilage-derived ECM powder.

### Experimental Example 1: Analysis of Components in ECM through Protein Array Method

Growth factor array was performed for component analysis in ECM powders of Preparation Example 2, Comparative Preparation Example 5, and Comparative Preparation Example 6.

As a specific method for screening components in ECM powder, ECM powder was lysed with a protein extraction solution, and then performed using Proteome Profiler Human Angiogenesis Array Kit capable of identifying 48 growth factors, and images were confirmed through Fluorescence Laser Scanner.

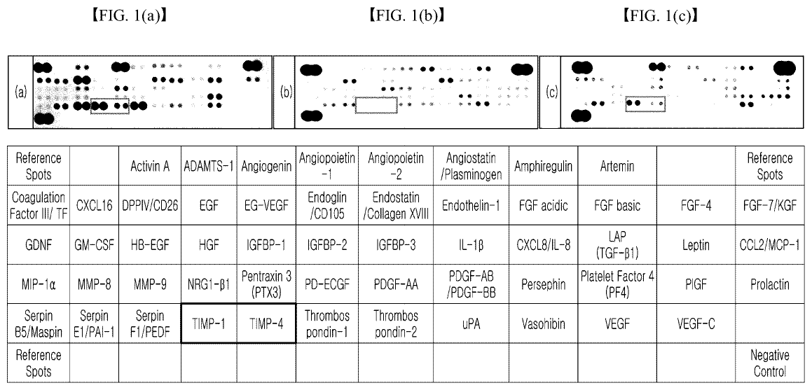

FIGS. 1(a), 1(b) and 1(c) are results of analyzing a total of 48 types of growth factors for ECM powders of Preparation Example 2, Comparative Preparation Example 5, and Comparative Preparation Example 6, respectively.

Referring to FIG. 1, it was confirmed that TIMP-1 and TIMP-4 proteins, known as nociceptive regulators, were contained in large amounts specifically only in the human tendon-derived ECM powder of Preparation Example 2.

### Experimental Example 2: Analysis of TIMP-1 Content in ECM

As it was confirmed through Experimental Example 1 that human tendon-derived ECM powder can contain a large amount of TIMP-1 protein, in order to confirm the effect of the manufacturing method of human tendon-derived ECM powder on TIMP-1 protein content, TIMP-1 content was quantified by ELISA method for human tendon-derived ECM powders of Comparative Preparation Examples 1 to 4 and Preparation Examples 1 to 3.

Specifically, each ECM powder was lysed with a protein extraction solution, and then performed using Human TIMP-1 ELISA Kit, and quantitative values were confirmed through Spectrophotometer.

FIG. 2 is a graph showing TIMP-1 content for human tendon-derived ECM powders of Comparative Preparation Examples 1 to 4 and Preparation Examples 1 to 3.

Referring to FIG. 2, it can be confirmed that the presence or absence of the defatting process affects the TIMP-1 content, and in particular, when the defatting process is performed on tendon tissue containing no moisture by freeze-drying, the remaining TIMP-1 content shows a remarkably high value of 20,000 pg/mg protein or more.

### Experimental Example 3: Analysis of Crude Fat Residual Amount

Crude fat residual amount was measured for human tendon-derived ECM powders of Comparative Preparation Examples 1 to 4 and Preparation Examples 1 to 3.

Specifically, ECM powder was put into a Soxhlet extractor, and after extraction, ethyl ether was circulated to extract ether soluble matter (mainly lipids) and collected in a receiver, and then ether was distilled off and dried, and the weight of the extract was weighed to quantify the content of crude fat.

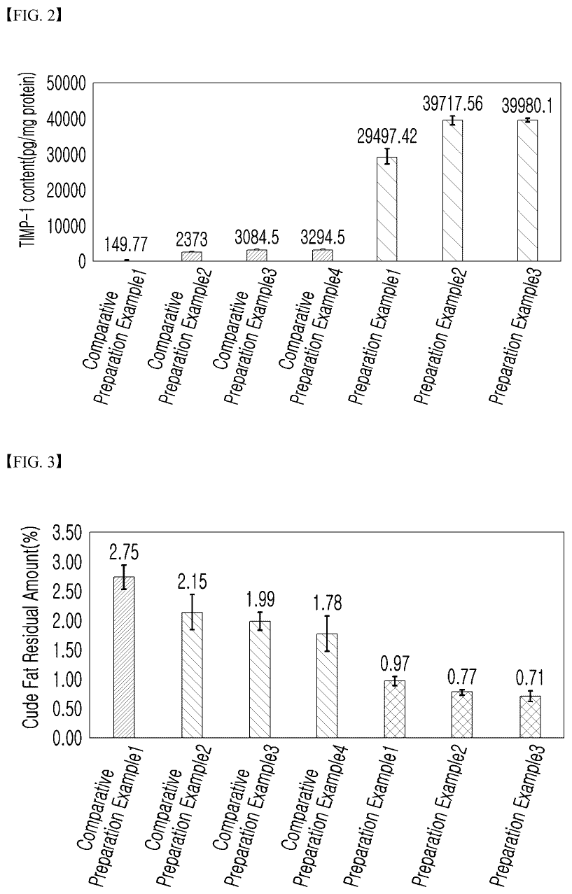

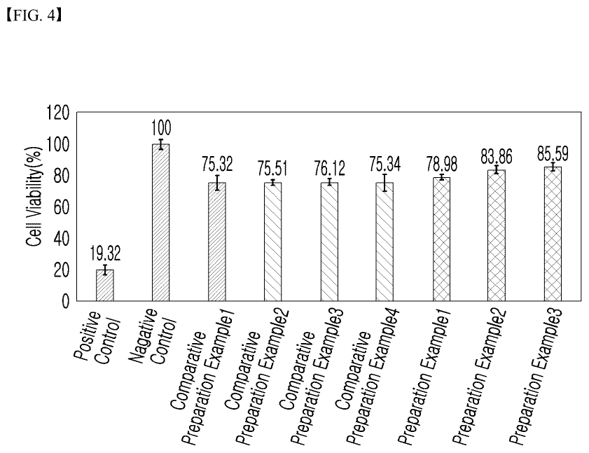

FIG. 3 is a graph showing crude fat residual amount for human tendon-derived ECM powders of Comparative Preparation Examples 1 to 4 and Preparation Examples 1 to 3.

Referring to FIG. 3, it can be confirmed that the presence or absence of the defatting process affects the residual amount of crude fat, and in particular, when the defatting process is performed on tendon tissue containing no moisture by freeze-drying, the crude fat content in the ECM powder is remarkably reduced to 1% by weight or less.

### Experimental Example 4: Cell Viability Test

Biological safety was tested for compositions comprising human tendon-derived ECM powders of Comparative Preparation Examples 1 to 4 and Preparation Examples 1 to 3.

Specifically, cells (NCTC clone 929) were prepared at a density of 1×10\hat{ }5 cells/ml in a 96 well plate and cultured in a 37° C., 5% CO2 incubator for 24 hours.

Each composition was eluted in a medium solution at 37° C. for 72 hours at a ratio of 20 ml per 4 g, and then cultured for 48 hours in a 37° C., 5% CO2 incubator using the composition eluate, blank test solution, negative and positive controls, and biological safety was evaluated by MTT method, and quantitative values were confirmed with Spectrophotometer.

FIG. 4 is a graph showing biological safety test results for compositions comprising human tendon-derived ECM powders of Comparative Preparation Examples 1 to 4 and Preparation Examples 1 to 3.

Referring to FIG. 4, it can be confirmed that biological safety significantly increases when the defatting process is performed on tendon tissue containing no moisture by freeze-drying.

### Experimental Example 5: Inflammation Relief Efficacy Test

Changes in expression of inflammation-related factors were evaluated before and after treatment with the composition comprising human tendon-derived ECM powder of Preparation Example 2 in human-derived fibroblast culture medium.

Specifically, human-derived fibroblasts (CCD-986sk) were cultured in a culture medium for proliferation for one day, and then the composition was treated at a concentration of 10% by weight.

After culturing cells for 3 days, the culture supernatant was obtained, and cytokine and chemokine expression of cells according to composition treatment was performed using Proteome Profiler Human Cytokine Array Kit, and images were confirmed through Fluorescence Laser Scanner.

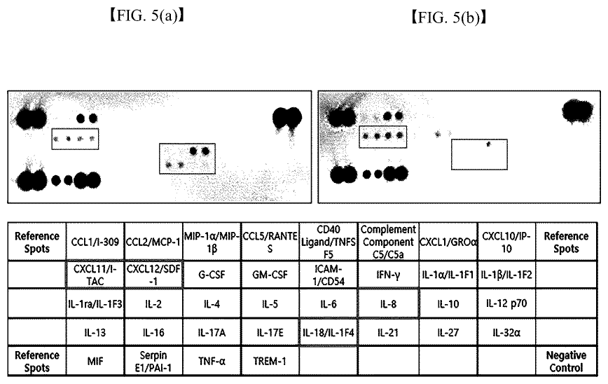

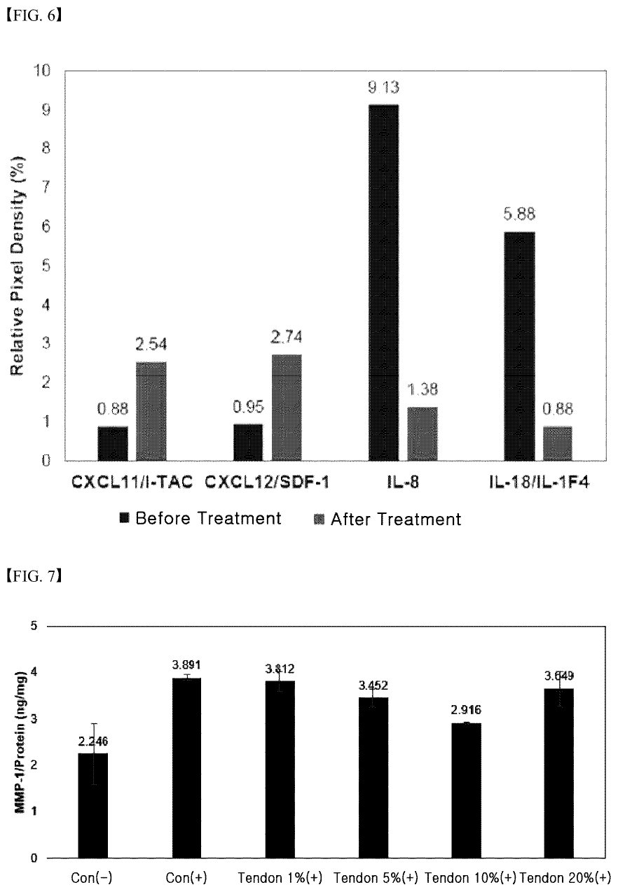

FIG. 5(a) and 5(b) are results of analyzing a total of 36 types of inflammation-related factors for human-derived fibroblast culture medium before and after treatment with the composition comprising human tendon-derived ECM powder of Preparation Example 2, respectively, and FIG. 6 is a graph illustrating changes in content of pro-inflammatory factors and anti-inflammatory factors before and after treatment with the composition comprising human tendon-derived ECM powder of Preparation Example 2.

IL-8 is a cytokine causing inflammation and is a pro-inflammatory factor, and IL-18 is a pro-inflammatory factor promoting production and expression of INF-y which is an inflammatory cytokine, and CXCL, a type of chemokine, is an anti-inflammatory factor (or factor aiding tissue repair).

Referring to FIGS. 5 and 6, after treatment with the composition comprising human tendon-derived ECM powder of Preparation Example 2, it can be confirmed that IL-8, a pro-inflammatory factor, decreased by 84.88%, IL-18 decreased by 85.03%, CXCL11, an anti-inflammatory factor, increased by 65.35%, and CXCL12 increased by 65.33%.

### Experimental Example 6: Pain Relief Efficacy Test

An inflammation model was established by treating (+) an inflammation inducing factor (IL-1β) in human-derived fibroblast culture medium, and then the composition comprising human tendon-derived ECM powder of Preparation Example 2 was treated to evaluate the degree of inhibition of pain-related proteins MMP-1 and MMP-9.

As a cell model, a pain-induced model was prepared by adding IL-1β known as a pro-inflammatory factor to human-derived fibroblasts (CCD-986sk) at a concentration of 10 ng/mL in a 6-well plate.

The composition was treated at 4 concentrations (1 wt %, 5 wt %, 10 wt %, 20 wt %) and cultured for 3 days, and then the expression levels of pain-related factors (MMP-1, MMP-3) were performed using Human MMP-1 ELISA Kit and Human MMP-3 ELISA Kit, and quantitative values were confirmed through Spectrophotometer.

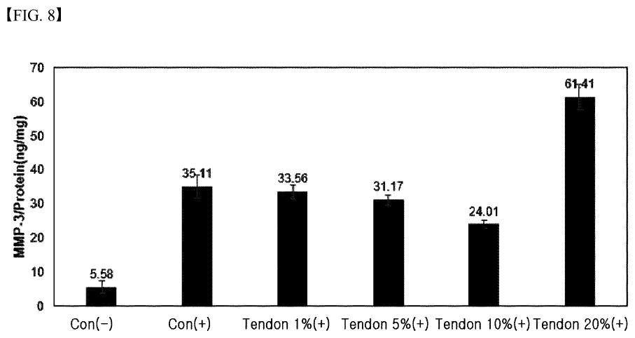

FIG. 7 is a graph showing a degree of inhibition on MMP-1, and FIG. 8 is a graph showing a degree of inhibition on MMP-3.

In FIGS. 7 and 8, Con(−) means the content of MMP-1 and MMP-3 contained in the human-derived fibroblast culture medium before treatment with the inflammation inducing factor, and Con(+) means the content of MMP-1 and MMP-3 contained in the human-derived fibroblast culture medium after treatment with the inflammation inducing factor.

From FIGS. 7 and 8, it can be confirmed that the tendon-derived ECM powder treatment group showed a significant decrease depending on the concentration from low concentration to 10% treatment group, and in particular, MMP-1 and MMP-3 decreased most effectively in the 10% treatment group.

The description of the present invention described above is for illustrative purposes, and those of ordinary skill in the art will readily appreciate that aspects of the present invention can easily be modified into other specific forms without changing the technical idea or essential features described in this specification.

Therefore, it should be understood that the embodiments described above are illustrative in all respects and not restrictive. For example, each component described as a single type may be implemented in a distributed form, and likewise components described as distributed may be implemented in a combined form.

The scope of the present invention is indicated by the following claims, and all changes or modifications derived from the meaning and scope of the claims and their equivalents should be construed as being included in the scope of the present invention.

1. A composition for preventing, ameliorating, or treating ligament or tendon diseases comprising tendon-derived Extracellular Matrix (ECM) powder as an active ingredient,
wherein a content of TIMP-1 protein contained in 1 mg of the tendon-derived ECM powder is 500 pg or more.

2. The composition of claim 1, wherein a crude fat content in the tendon-derived ECM powder is 2.0% by weight or less based on 100% by weight of the tendon-derived ECM powder.

3. The composition of claim 1, wherein an average particle diameter of the tendon-derived ECM powder is 425 μm or less.

4. The composition of claim 1, wherein the tendon-derived ECM powder is included in an amount of 1 to 99% by weight relative to 100% by weight of the total composition.

5. The composition of claim 1, wherein the tendon-derived ECM powder is manufactured by a method comprising the steps of:
(a) collecting tendon tissue;
(b) decellularizing the tendon tissue to obtain tendon-derived ECM;
(c) freeze-drying the tendon-derived ECM;
(d) defatting the freeze-dried tendon-derived ECM; and
(e) degassing the defatted tendon-derived ECM, followed by freeze-grinding to obtain tendon-derived ECM powder.

6. The composition of claim 1, further comprising a pharmaceutically acceptable carrier.

7. The composition of claim 1, further comprising one or more biocompatible polymers selected from the group consisting of hyaluronic acid, polynucleotide, polydeoxyribonucleotide, carboxymethyl cellulose, collagen, chitosan, alginate, and gelatin.

8. The composition of claim 7, wherein the biocompatible polymer is cross-linked with one or more cross-linking agents selected from the group consisting of 1,4-butanediol diglycidyl ether (BDDE), ethylene glycol diglycidyl ether (EGDGE), 1,6-hexanediol diglycidyl ether, propylene glycol diglycidyl ether, polypropylene glycol diglycidyl ether, polytetramethylene glycol diglycidyl ether, neopentyl glycol diglycidyl ether, and 1,2-(bis(2,3-epoxypropoxy)ethylene).

9. The composition of claim 1, wherein the composition has a pH of 5.0 to 8.0.
